(19) United States
(12) Patent Application Publication
(10) Pub. No.: US 2023/0404565 A1
(43) Pub. Date: Dec. 21, 2023

(54) DEVICES, SYSTEMS, AND METHODS FOR DELIVERING AND DEPLOYING AN IMPLANTABLE DEVICE

(71) Applicants: BOSTON SCIENTIFIC SCIMED, INC., MAPLE GROVE, MN (US); Mayo Foundation for Medical Education and Research, Rochester, MN (US)

(72) Inventors: Daniel Shuey, Pine City, MN (US); Joel T. Eggert, Plymouth, MN (US); Mitchell Nelson, Hudson, WI (US); Eric Jason Krause, Big Lake, MN (US); Evan M Leingang, Plymouth, MN (US); Greg Johnson, Minneapolis, MN (US); Greg Hoepfner, Brooklyn Park, MN (US)

(73) Assignees: BOSTON SCIENTIFIC SCIMED, INC., MAPLE GROVE, MN (US); Mayo Foundation for Medical Education and Research, Rochester, MN (US)

(21) Appl. No.: 18/211,542

(22) Filed: Jun. 19, 2023

Related U.S. Application Data
(60) Provisional application No. 63/354,005, filed on Jun. 21, 2022.

(51) Int. Cl. A61B 17/04; A61B 17/00

(52) U.S. Cl. CPC A61B 17/0401; A61B 17/00234; A61B 17/0487; A61B 2017/0409; A61B 2017/0496; A61B 2017/00243

(57) ABSTRACT

A delivery and deployment system having a control handle operable to deliver and/or deploy an implantable device, e.g., transluminally. A steering control system may include a steering control knob rotatable about the longitudinal axis of the handle to axially pull on a steering element to steer a flexible elongate member to deliver an implantable device to a desired site. An implantable device deployment system may include a slider selectively slidable with respect to the control handle to deploy an implantable device, and optionally to retain the implantable device in a delivery position. A tether adjustment system may include a knob rotatable about the handle's longitudinal axis to axially pull on a tether element to adjust the tension and/or length thereof. A tensioning and locking system may include a knob rotatable about the handle's longitudinal axis to shift a tensioning and locking device to set the tether element's tension and/or length.

### CROSS-REFERENCE TO RELATED APPLICATIONS

This application claims the benefit of priority under 35 U.S.C. § 119 to U.S. Provisional Application No. 63/354,005, filed Jun. 21, 2022, the entire disclosure of which is hereby incorporated by reference herein for all purposes.

### FIELD

The present disclosure relates generally to the field of medical devices and systems. More particularly, the present disclosure relates to devices, systems, and methods for delivering and deploying implantable devices. Even more particularly, the present disclosure describes devices, systems, and methods for steering elongate members such as catheters, and for deploying implantable devices such as tissue-engaging elements (such as tissue anchors), and for adjusting tension on an element operatively associated with the tissue-engaging element.

### BACKGROUND

Devices, systems, and methods for delivering and/or deploying medical devices with minimally invasive techniques, such as percutaneously and/or transluminally, are desirable for avoiding more complex and invasive open surgical procedures. Various techniques which do not require open surgery utilize systems and devices with various flexible elongate members capable of navigating to an anatomical site within the body from a small insertion opening in a patient's body or through a natural orifice; transluminally through the body (such as through the vascular system); and to an anatomical site. To reach an anatomical site within the body, various elongate members must be steerable to navigate through tortuous pathways within the body leading to the anatomical site. Improvements to handles for steering elongate members would be welcome in the art. Moreover, to deploy an implantable device inserted percutaneously and/or transluminally, the delivery/deployment system must provide proper control of movement of the implantable device as well as proper control of any system for delivering and deploying the implantable device within the patient's body. Improvements to handles for delivering and deploying an implantable device, and/or adjusting any further devices operatively associated therewith, would be welcome in the art.

### SUMMARY

This summary of the disclosure is given to aid understanding, and one of skill in the art will understand that each of the various aspects and features of the disclosure may advantageously be used separately in some instances, or in combination with other aspects and features of the disclosure in other instances. No limitation as to the scope of the claimed subject matter is intended by either the inclusion or non-inclusion of elements, components, or the like in this summary.

In accordance with various principles of the present disclosure, a delivery and deployment system for delivering and/or deploying an implantable device to an anatomical site, includes a flexible elongate member; an implantable device delivery device deliverable at a distal end of the flexible elongate member to an anatomical site; an implantable device deliverable by the implantable device delivery device; and a control handle. In some aspects, the control handle includes one or more of the following: a steering system operably coupled with the flexible elongate member to control movement of the flexible elongate member to deliver the implantable device to the anatomical site; an implantable device deployment system operably coupled with the implantable device to deploy the implantable device at the anatomical site; a tether adjustment system operably engageable with a tether element engageable with the implantable device; or a tensioning and locking system operably engageable with the implantable device to adjust a configuration of a tensioning and locking device operably associated with the implantable device.

In some embodiments, the flexible elongate member includes a pull element operatively associated therewith; and the control handle includes at least a steering system, the steering system including a steering control knob rotatable about the longitudinal axis of the control handle to pull the pull element axially to steer the flexible elongate member.

In some embodiments, the implantable device is a tissue anchor.

In some embodiments, the flexible elongate member is tubular, and the delivery and deployment system further includes a stylet operably coupled with the implantable device and extending through the tubular flexible elongate member. In some embodiments, the control handle includes at least an implantable device deployment system operably coupled with the stylet to axially translate the stylet along the longitudinal axis of the control handle to advance and to deploy the implantable device from the implantable device delivery device.

In some embodiments, the system further includes a tether element extending with respect to the implantable device to a location spaced from the implantable device, and the control handle includes at least a tether adjustment system operably engageable with the tether element to adjust the tension on and/or length of the tether element between the implantable device and the location spaced from the implantable device. In some embodiments, the tether adjustment system includes a fine tuning knob engageable with the tether element and rotatable about the longitudinal axis of the control handle to adjust the tension on and/or length of the tether element between the implantable device and the location spaced from the implantable device. In some embodiments, the system further includes a tensioning and locking device operably associated with the tether element and shiftable from a tension-adjusting configuration, in which tension on and/or the length of the tether element is adjustable, to a tension-setting configuration, in which tension on and/or the length of the tether element is fixed with respect to the implantable device. In some embodiments, the control handle further comprises a tensioning and locking system operably associated with the tensioning and locking device to shift the tensioning and locking device between the tension-adjusting configuration and the tension-setting configuration. In some embodiments, the tensioning and locking system includes a tensioning and locking knob rotatable about the longitudinal axis of the control handle to shift the tensioning and locking device between the tension-adjusting configuration and the tension-setting configuration. In some embodiments, the tensioning and locking system further includes an anchor state indicator operably associated with the tensioning and locking knob to indicate the configuration of the tensioning and locking device. In some embodiments, the system further includes a stylet operably coupled with the implantable device. In some embodiments, the control handle comprises at least an implantable device deployment system operably coupled with the stylet to axially translate the stylet along the longitudinal axis of the control handle to advance and to deploy the implantable device from the implantable device delivery device. In some embodiments, the stylet is operably coupled with the tensioning and locking device and the tensioning and locking system such that rotation of the tensioning and locking knob rotates the stylet, and rotation of stylet shifts the tensioning and locking device between the tension-adjusting configuration and the tension-setting configuration.

In some embodiments, the system further includes a tether element and a tensioning and locking device operably associated with the tether element; the tether element extends from the implantable device to a location spaced from the implantable device; the tensioning and locking device is shiftable from a tension-adjusting configuration, in which tension on and/or the length of the tether element is adjustable, to a tension-setting configuration, in which tension on and/or the length of the tether element is fixed with respect to the implantable device; and the control handle includes at least the tensioning and locking device to shift the tensioning and locking device between the tension-adjusting configuration and the tension-setting configuration.

In accordance with various principles of the present disclosure, a delivery and deployment system for delivering and/or deploying an implantable device to an anatomical site includes a control handle having a longitudinal axis and comprising at least one control knob rotatable about the longitudinal axis of the control handle and operable to axially translate a component of the delivery and deployment system.

In some embodiments, the delivery and deployment system includes a flexible elongate member configured to deliver an implantable device to an anatomical site transluminally; and a pull element is operably coupled with the flexible elongate member and the control knob such that rotation of the control knob about the longitudinal axis of the control handle bends a distal region of the flexible elongate member to deliver the implantable device to a treatment site.

In some embodiments, the control knob is rotatable about the longitudinal axis of the control handle to adjust an element of the delivery and deployment system with respect to a tether element operably associated with an implantable device deliverable by the delivery and deployment system.

In accordance with various principles of the present disclosure, a method of delivering and/or deploying an implantable device transluminally includes rotating a control knob operably associated with a delivery and deployment system control handle about the longitudinal axis of the control handle to axially translate an element of a delivery and deployment system associated with an implantable device.

In some embodiments, the axially translated element is at least one of a pull element operably associated with a steerable flexible elongated member and axially translatable to bend the steerable flexible elongate member to steer the steerable flexible elongate member within a patient's body to deliver an implantable device operably associated with the steerable flexible elongate member, or a tether element operably associated with an implantable device, or a flexible elongate member operably associated with a tether element and an implantable device to adjust tension on and/or the length of the tether element with respect to the implantable device.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates a perspective view of an example of an embodiment of a delivery and deployment system and handle in accordance with various aspects of the present disclosure.

FIG. 2 illustrates a perspective view of an example of an embodiment of an implantable device delivery and deployment system positioned, by a delivery and deployment system formed in accordance with various principles of the present disclosure, to deploy an implantable device with respect to a schematic representation of a heart.

FIG. 3 illustrates an enlarged isolated perspective view of an implantable device delivery and deployment system control handle as illustrated in FIG. 1.

FIG. 4 illustrates a perspective view similar to that of FIG. 3, but with a portion of the handle housing removed.

FIG. 5 illustrates a cross-sectional view along line V-V of FIG. 3.

FIG. 6 illustrates a perspective view of an example of an embodiment of an implantable device deployed with respect to a schematic representation of a heart by a delivery and deployment system formed in accordance with various principles of the present disclosure.

FIG. 7 illustrates a perspective view of an implantable device with a housing portion thereof in phantom to reveal a tensioning and locking device therein, deliverable and deployable by a delivery and deployment system formed in accordance with principles of the present disclosure.

FIG. 8A illustrates a cross-sectional view along line VIIIA-VIIIA of an artificial chordae tendineae tensioning and locking device as in FIG. 7 in an unlocked configuration.

FIG. 8A illustrates is a cross-sectional view of an artificial chordae tendineae tensioning and locking device similar to that of FIG. 8A, but shown in a locked configuration.

FIG. 8C illustrates is a cross-sectional view similar to that of FIG. 8B and FIG. 8C, showing an actuator formed in accordance with principles of the present disclosure disengaged from the artificial chordae tendineae tensioning and locking device.

FIG. 9A illustrates a detail view of a tensioning and locking system as in FIG. 4, with a tensioning and locking knob in a position corresponding to the position of the tensioning and locking device illustrated in FIG. 8A.

FIG. 9B illustrates a perspective view similar to that of FIG. 9A, but with the tensioning and locking knob in a position corresponding to the position of the tensioning and locking device illustrated in FIG. 8B.

FIG. 9C illustrates a perspective view similar to that of FIG. 9A, but with the tensioning and locking knob in a position corresponding to the position of the tensioning and locking device illustrated in FIG. 8C.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a strut, a channel, a cavity, or a bore. As used herein, a “channel” or “bore” is not limited to a circular cross-section. As used herein, a “free end” of an element is a terminal end at which such element does not extend beyond.

In accordance with various principles of the present disclosure, a delivery and deployment system is configured to deliver and/or deploy an implantable device with respect to a treatment site. It will be appreciated that reference is made herein to delivery “and” deployment for the sake of convenience without intent to limit. In some aspects, the delivery and deployment system is configured to operate and/or includes an implantable device delivery and deployment system configured to deliver and deploy an implantable device with respect to tissue at an anatomical site, such as a treatment site within a patient's body, and optionally with respect to another anatomical site. It will be appreciated that reference may be made herein to an anatomical site, delivery site, deployment site, implant/implantation site, site of implantation, target site, treatment site, etc., interchangeably and without intent to limit. In some aspects, the delivery and deployment system includes a handle configured to operate such implantable device deployment system to deploy the associated implantable device. In some embodiments, the implantable device is a tissue-engaging element such as a tissue anchor configured to be deployed to be implanted into tissue. It will be appreciated that terms such as implant (and other grammatical forms thereof) may be used interchangeably herein with terms (and grammatical forms thereof) such as affix, anchor, attach, associate, couple, engage, embed, hold, retain, purchase, secure, etc., without intent to limit.

In an example of an embodiment of a delivery and deployment system described herein, the system includes a handle configured to facilitate steering of an implantable device delivery and deployment system. For example, the handle may include a steering knob rotatable to actuate a pull element to direct the implantable device delivery and deployment system to an anatomical site at which the implantable device is to be deployed. It will be appreciated that terms such as steer (and other grammatical forms thereof) may be used interchangeably herein with such terms (and other grammatical forms thereof) actuate, advance, articulate, bend, control, drive, flex, manipulate, maneuver, move, navigate, operate, pull, retract, rotate, shift, transition, translate, turn, etc., without intent to limit.

It will be appreciated that to deliver an implantable device transluminally, the implantable device may be shiftable between a delivery configuration and an expanded deployed configuration. The implantable device may be delivered transluminally within a tubular element, such as to retain the implantable device in a delivery configuration and/or to protect the implantable device, and/or to protect the body passage through which the implantable device is advanced. The tubular element may be delivered by a flexible elongate member. It will be appreciated that the term flexible elongate member or tubular flexible elongate member is used herein generically to refer to elements such as catheters, shafts, cannulas, sheaths, tubes, stylets, etc., for the sake of convenience and without intent to limit. Once at a treatment site, the implantable device may be advanced distally out of the tubular element to be deployed. For the sake of convenience, and without intent to limit, the implantable device is referenced herein as an anchor, and the tubular element in which the anchor is delivered (generally without an additional intervening tubular delivery element therebetween) is referenced herein as an anchor garage, although principles of the present disclosure are applicable to implantable devices other than anchors and tubular elements other than anchor garages. The anchor garage may be specifically configured to retain the anchor therein. Optionally, the anchor garage has additional features facilitating use of the anchor garage with other devices or systems operable in conjunction with the anchor. For instance, if the anchor is to be used in conjunction with another device, then the anchor garage may include features allowing cooperation of the anchor and/or anchor garage with such other devices or systems. Such an anchor garage may be mounted at a distal end of a tubular flexible elongate member which may be referenced as a garage shaft for the sake of convenience and without intent to limit. The anchor may be coupled to a flexible elongate member to be movable with respect to the anchor garage, such as to be deployed therefrom. For the sake of convenience, and without intent to limit, the flexible elongate member with which the anchor is coupled is referenced herein as an anchor stylet.

In an example of an embodiment of a delivery and deployment system described herein, the system includes a handle with a slider operable to deploy the anchor from a delivery configuration, within an anchor garage, into a deployed configuration outside the anchor garage, such as within tissue. In some embodiments, the slider is operatively coupled with the anchor stylet to distally advance the anchor out of the anchor garage to deploy the anchor. In accordance with various principles of the present disclosure, the slider may be locked in place to prevent inadvertent unintentional movement of the anchor.

As may be appreciated, the delivery and deployment system may be subjected to compressive forces during transluminal navigation to the treatment site. In some embodiments, the slider may be actuated to apply tension to at least one component of the system so that the anchor is not prematurely deployed as a result of forces on the system. For instance, in some embodiments, the anchor garage and/or the garage shaft may be subjected to compressive forces which may cause the anchor garage to retract relative to the anchor and prematurely deploy or at least partially deploy the anchor. In some embodiments, the slider may be movable to selectively apply tension to the anchor stylet to maintain the anchor within the anchor garage and to prevent premature deployment thereof. Such ability to maintain the anchor within the anchor garage may be particularly beneficial if the anchor automatically shifts from a delivery configuration to a deployed configuration, such as by expanding upon exiting the anchor garage.

In some aspects, an implantable device such as a tissue anchor may be coupled with another implantable device and/or a tether element may be coupled to the tissue anchor. For instance, a tether element may be operatively coupled between a first implantable device and a second implantable device. In some embodiments, a tether element is operably associated with the implantable device at a first location at an anatomical site and operably associated with a second location spaced apart from the first location. For instance, the tether element may be coupled with another implantable device deployed spaced apart from the initial implantable device. For instance, the tether element may extend between the implantable tissue-engaging element and another tissue-engaging device such as a clip or clamp. The tether element may be attached to one of the tissue-engaging element or the tissue-engaging device in a generally fixed manner, and movably coupled with another element or device. In the illustrated example of an embodiment of an implantable device and associated delivery and deployment devices and systems described herein, the implantable device is in the form of a tissue anchor coupled via a suture to a tissue clip or clamp. More particularly, in the illustrated example of an embodiment described herein, the tissue anchor is an anchor configured to be implanted in cardiac tissue (e.g., papillary muscle tissue), the suture is an artificial chordae tendineae, and the tissue clip is a leaflet clip. For the sake of convenience, and without intent to limit, a tether element may be alternately referenced herein as a tether or suture or artificial chordae tendineae without intent to limit. Moreover, for the sake of convenience and without intent to limit, the second implantable device to which the anchor is couple is referenced herein as a clip (or leaflet clip). It will be appreciated that principles of the present disclosure may nonetheless be applicable to devices other than cardiac tissue anchors, leaflet clips, and/or sutures or artificial chordae tendineae.

In some instances, it may be desirable to control movement of the tether with respect to the implantable device, such as to apply tension, via the tether, to the implantable device and/or between the first implantable device and the second implantable device. In some embodiments, the tether is coupled to one of the implantable devices and movable with respect to the other of the implantable devices. In some embodiments, the implantable device to which the tether is coupled is implanted first, and the other of the implantable devices is implanted second, and the tether may be pulled proximally (e.g., through the delivery and deployment system and relative to the handles thereof), relative to the other of the implantable devices to adjust the length and/or tension of the tether between the implantable devices. Such adjustment of the tether may be considered a gross adjustment, as it is generally not a measured and precisely-controlled adjustment.

In various anatomical sites, precise, fine adjustment of the tether is necessary. For instance, in the illustrated example of an embodiment described herein, the first implantable device is a cardiac tissue anchor and the second implantable device is a leaflet clip. The leaflet clip is clamped onto a heart valve leaflet, and then the tissue anchor is anchored with respect to a heart ventricle, with an artificial chordae tendineae coupled to the leaflet clip and extending to the tissue anchor. The artificial chordae tendineae is movable with respect to the tissue anchor to adjust tension on the leaflet clip to restore proper functioning to the leaflet (e.g., to prevent the leaflet from extending or flailing into the atrium and thus preventing the heart valve from closing properly, which may result in regurgitation and various deleterious consequences). To achieve proper closure of the heart valve, the tension on the heart valve leaflet must be sufficient enough to prevent flailing, but not so high as to prevent the leaflet from returning to the closed position once blood flows from the atrium to the ventricle.

In accordance with various principles of the present disclosure, a delivery and deployment system includes a tether control knob for fine adjustment of the tether length and/or tension. A tether lock may be provided to lock the tether with respect to the tether control knob. The tether lock may be shiftable between a disengaged position in which the tether may move relatively freely with respect to the tether control knob, and an engaged position in which the tether is locked with respect to the tether control knob. In some embodiments, the tether control knob is in the form of a rotatable knob controlling axial movement of the tether. When the rotatable tether control knob is rotated in a first direction, the tether is pulled proximally to add tension on the tether and/or to shorten the length of the tether within the anatomical site. When the rotatable tether control knob is rotated in a second direction (opposite the first direction), tension on the tether is reduced or the tether is paid out to increase the length of the tether within the anatomical site (such as by the leaflet pulling on the tether to increase the length of the tether between the leaflet clip and the anchor, and/or to decrease tension therebetween).

It will be appreciated that once the desired tension on and/or length of the tether is reached or achieved, it may be desirable to set or fix or lock such configuration. In an example of an embodiment of a delivery and deployment system described herein, the system includes a tensioning and locking device operably associated with the tether to set or fix the desired tether tension and/or length. In some embodiments, the tensioning and locking device adjusts the position of the tether with respect to the implantable device, and/or tension on one or both implantable devices, and/or the length of the tether extending between the implantable devices. The tensioning and locking device may be shiftable between a tension-adjusting configuration and a tension-setting or locking configuration. In the tension-adjusting configuration, the tensioning and locking device allows movement of the tether relative to the tensioning and locking device. such as to adjust tension of the tether with respect to the clip and/or anchor. For instance, a locking element may be in a position which is not engaged with/disengaged from the tether to allow free movement of the tether. In the tension-setting or locking configuration (such terms being used interchangeably herein without intent to limit), the tensioning and locking device inhibits movement of the tether, such as relative to the tensioning and locking device, to set the length and/or tension of the tether. For instance, a locking element may be shifted into engagement with the tether to inhibit or prevent movement of the tether with respect to the tensioning and locking device. It will be appreciated that terms such as inhibit, prevent, lock, limit, etc., including other grammatical forms thereof, may be used interchangeably herein without intent to limit. The tensioning and locking device may be shifted into the tension-locking configuration once a desired position of the tether with respect to the implantable device, and/or length of the tether extending between the implantable devices, and/or tension on one or both implantable devices has been achieved.

In accordance with various principles of the present disclosure, a delivery and deployment system control handle includes a tensioning and locking knob actuatable to adjust the configuration of a tensioning and locking device to adjust the tension on and/or length of a tether element associated with one or more implantable devices. In some embodiments, the anchor stylet is operably coupled to a component of the tensioning and locking device to shift the tensioning and locking device between the tension-adjusting configuration and the tension-setting configuration. In some embodiments, the stylet is operably coupled with a movable locking element which is shifted with respect to the tether to shift the configuration of the tensioning and locking device. In some embodiments, the stylet is rotatable to shift the configuration of the tensioning and locking device. In some embodiments, rotation of the stylet moves the locking element axially with respect to the housing to shift the configuration of the tensioning and locking device. In such embodiments, rotation of the stylet control knob rotates the stylet to shift the tensioning and locking device between the tension-adjusting configuration and the tension-setting configuration. More particularly, in some embodiments, rotation of the stylet causes generally linear translation of a movable locking element with respect to the tether to fix the tether with respect to the anchor and/or to allow adjustment of the tether with respect to the anchor (e.g., to adjust the length of and/or tension on the tether with respect to the anchor and the leaflet clip). In some embodiments, rotation of the stylet a sufficient amount decouples the stylet from operable engagement with the anchor and/or the tensioning and locking device, such as to release and deploy the implantable device. In some embodiments, the tensioning and locking device is operably associated with the implantable device. In some embodiments, the tensioning and locking device is housed in a housing coupled to the implantable device. It will be appreciated that principles of the present disclosure are applicable to set tension on a tether whether or not a stylet is used

In some embodiments, an indicator is provided to indicate the status of the implantable device and/or the tensioning and locking device such as relative to or correlated with the position of the tensioning and locking knob. For instance, the indicator may have indicia indicative of whether the tensioning and locking device is in a tension-adjusting or tension-setting configuration, and/or whether the stylet is operably engaged with the implantable device or has been decoupled/dissociated therefrom.

It will be appreciated that the devices, systems, and methods of the present disclosure may be used alone or together with other devices, systems, and methods to treat heart disease. Examples of devices, systems, and methods with which embodiments of the present disclosure may be implemented include, but are not limited to, those described in U.S. Patent Application Publication US2021/0007847, titled Devices, Systems, And Methods For Clamping A Leaflet Of A Heart Valve, and published on Jan. 14, 2021; U.S. Patent Application Publication US2021/0000597, titled Devices, Systems, And Methods For Adjustably Tensioning An Artificial Chordae Tendineae Between A Leaflet And A Papillary Muscle Or Heart Wall, and published on Jan. 7, 2021; U.S. Patent Application Publication US2021/0000598, titled Devices, Systems, And Methods For Anchoring An Artificial Chordae Tendineae To A Papillary Muscle Or Heart Wall, and published on Jan. 7, 2021; U.S. Patent Application Publication US2021/0000599, titled Devices, Systems, And Methods For Artificial Chordae Tendineae, and published on Jan. 7, 2021; U.S. Patent Application Publication 2022/0096235, titled Devices, Systems, And Methods For Adjustably Tensioning Artificial Chordae Tendineae In A Heart, and published on Mar. 31, 2022; U.S. Patent Application Publication 2023/0062599 titled Devices, Systems, And Methods For Anchoring An Artificial Chordae Tendineae To Cardiac Tissue, and published on Mar. 2, 2023; and U.S. Patent Application ______, filed Dec. 20, 2021 [ATTORNEY DOCKET 2001.2715100, FORMERLY 8150.0817Z], each of which is herein incorporated by reference in its entirety and for all purposes. Examples of devices described therein may be modified to incorporate embodiments or one or more features of the present disclosure.

Various embodiments of delivery and deployment system formed in accordance with various principles of the present disclosure will now be described with reference to examples of embodiments illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. The following description is of illustrative examples of embodiments only, and is not intended as limiting the broader aspects of the present disclosure.

Turning now to the drawings, it will be appreciated that common features are identified by common reference elements and, for the sake of brevity and convenience, and without intent to limit, the descriptions of the common features are generally not repeated. For purposes of clarity, not all components having the same reference number are numbered. Moreover, a group of similar elements may be indicated by a number and letter, and reference may be made generally to one or such elements or such elements as a group by the number alone (without including the letters associated with each similar element). Moreover, certain features in one embodiment may be used across different embodiments and are not necessarily individually labeled when appearing in different embodiments.

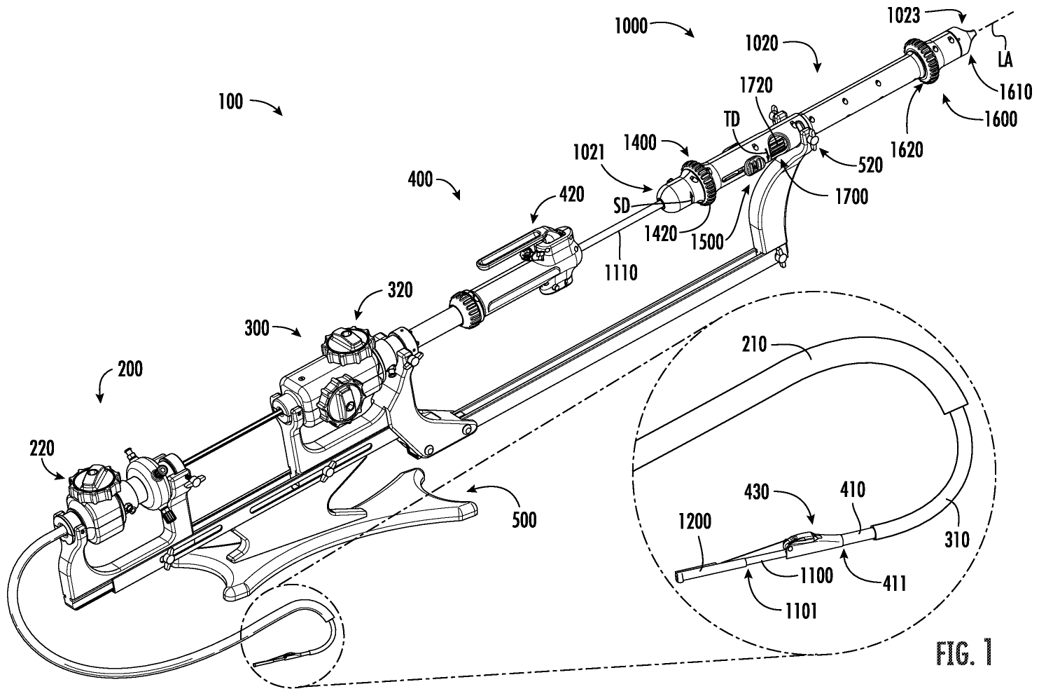

Turning now to the drawings, an example of an embodiment of a delivery and deployment system 1000 formed in accordance with various principles of the present disclosure is illustrated in FIG. 1. The delivery and deployment system 1000 optionally is part of a larger delivery/deployment system 100, such as described in further detail in in co-pending provisional patent application ______, [ATTORNEY DOCKET 2001.2787100], titled Devices, Systems, And Methods For Steering A Catheter, and filed on Jun. 21, 2022; and co-pending provisional patent application ______, [ATTORNEY DOCKET 2001.2754100], titled Devices, Systems, And Methods For Deploying An Implantable Device, and filed on Jun. 21, 2022; each of which application is incorporated by reference herein in its entirety and for all purposes. The illustrated example of an embodiment of the larger delivery/deployment system 100 includes a delivery system 200 having an introducer shaft 210 (which may also be referenced as a delivery sheath, without intent to limit) configured to deliver and introduce devices, systems, and components of the delivery/deployment system 100 to a treatment area. As such, the introducer shaft 210 is a tubular flexible elongate member defining a lumen therethrough sized, shaped, configured, and/or dimensioned to allow passage of further devices and/or systems therethrough for delivery to the treatment site, such as devices and/or systems delivered/deployed by the delivery and deployment system 1000. As referenced herein, a treatment area is an area on or within a patient's body at which treatment site is located. As referenced herein, a treatment site is an anatomical site at which a procedure is to be performed with the use of systems and/or devices delivered by the delivery/deployment system 100, and the delivery and deployment system 1000. It will be appreciated that reference to “at” the treatment site is intended to include at or about the vicinity of (e.g., along, adjacent, etc.) the treatment site, and is not limited to just the exact site of treatment. The introducer shaft 210 may be steerable, such as at least one-way or two-way steerable within a steering plane, such as with the use of a control handle 220 operatively associated therewith.

The delivery/deployment system 100 may include an additional steerable delivery system 300 having a steerable flexible elongate member 310 configured to provide further steering capabilities to the delivery/deployment system 100 to guide and direct devices and/or systems more closely to the desired treatment site, as may be appreciated with reference to the detail view in FIG. 1. The steerable flexible elongate member 310 may be tubular, defining a lumen therethrough sized, shaped, configured, and/or dimensioned to allow passage of further devices and/or systems therethrough for delivery to the treatment site, such as devices and/or systems delivered/deployed by the delivery and deployment system 1000, and thus is reference herein as a steerable “tubular” flexible elongate member to differentiate from other flexible elongate members described herein. The steerable tubular flexible elongate member 310 may be at least one-way steerable or two-way steerable within a steering plane, four-way within two transverse steering planes, etc., such as with the use of a control handle 320 operatively associated therewith. It will be appreciated that further details of an example of an embodiment of a delivery system 200 and an example of an embodiment of a steerable delivery system 300 are provided in above-incorporated co-pending provisional patent application ______, [ATTORNEY DOCKET 2001.2787100], titled Devices, Systems, And Methods For Steering A Catheter, and filed on Jun. 21, 2022. However, the present disclosure need not be limited by the examples of embodiments disclosed therein.

Optionally, the delivery/deployment system 100 includes an additional device delivery and deployment system 400 having a flexible elongate member 410 delivered through the steerable tubular flexible elongate member 310 and the introducer shaft 210 to the deployment site. Although the additional device delivery and deployment system 400 may include a control handle 420 to control operation thereof, the steerable delivery system 300 (such as via the steerable tubular flexible elongate member 310) may be used to steer a deployment system 430 mounted at a distal end 411 of the flexible elongate member 410 to a desired position with respect to the deployment site. In some embodiments, the deployment system 430 may be larger than the lumen of the steerable tubular flexible elongate member 310 and therefore may be delivered outside and distal to the steerable tubular flexible elongate member 310, as illustrated in the detail view in FIG. 1. It will be appreciated that further details of an example of an embodiment of a delivery and deployment system 400 are provided in above-incorporated co-pending provisional patent application ______, [ATTORNEY DOCKET 2001.2754100], titled Devices, Systems, And Methods For Deploying An Implantable Device, and filed on Jun. 21, 2022. However, the present disclosure need not be limited by the examples of embodiments disclosed therein.

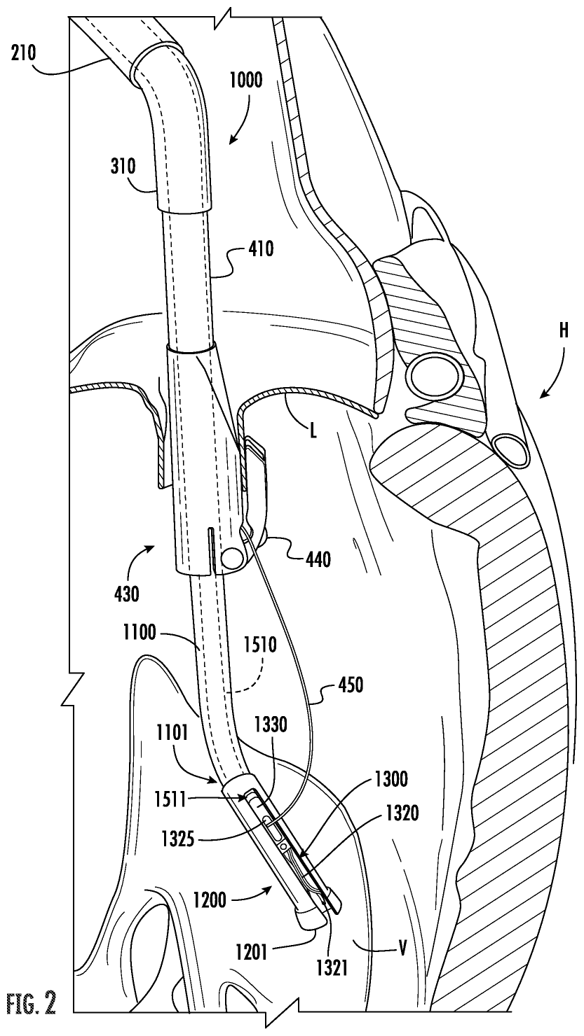

In accordance with various principles of the present disclosure, the delivery and deployment system 1000 may deliver, operate, and/or deploy an implantable device, such as a tissue anchor, to an anatomical site. In the example of an embodiment of a delivery and deployment system illustrated in FIG. 1 (as may be more readily seen in the detail view thereof) and in FIG. 2, the delivery and deployment system 1000 includes a flexible elongate member 1100 which delivers an implantable device delivery device 1200 configured to deliver an implantable device 1300. In the illustrated example of an embodiment, the implantable device delivery device 1300 is a generally tubular anchor garage 1200 with an implantable device in the form of a tissue anchor 1300 delivered therein. It will be appreciated that the implantable device delivery device 1300 may be in other forms or configurations, the present disclosure not being limited to an anchor garage. However, reference is made herein to an anchor garage for the sake of convenience and without intent to limit. The anchor garage 1200 may be mounted on a distal end 1101 of the flexible elongate member 1100 (which may alternately be referenced as a garage shaft), and optionally is not positioned within the steerable tubular flexible elongate member 310. If an additional deployment system 430 is delivered with the assistance of the steerable tubular flexible elongate member 310, and is delivered outside and distal to the steerable tubular flexible elongate member 310, the anchor garage 1200 may optionally be positioned outside and distal to such additional deployment system 430.

The flexible elongate member 1100 of the delivery and deployment system 1000 may be delivered within the introducer shaft 210 and the steerable tubular flexible elongate member 310 of the delivery/deployment system 100, with the anchor garage 1200 within the introducer shaft 210, or outside the introducer shaft 210 as described above. The introducer shaft 210 delivers the anchor garage 1200 to a general treatment area, such as a heart ventricle in the example of an embodiment illustrated in FIG. 2. The steerable tubular flexible elongate member 310 may further guide and direct the flexible elongate member 1100 of the delivery and deployment system 1000 to deliver the anchor garage 1200 to the deployment site at which the anchor 1300 carried therein is to be deployed. For instance, as illustrated in FIG. 2, the steerable tubular flexible elongate member 310 is steerable to direct the flexible elongate member 1100 towards a ventricle wall.

In the example of an embodiment of a delivery and deployment system 1000 illustrated in FIG. 1, a delivery and deployment system control handle 1020, operatively associated with the flexible elongate member 1100, may be translated generally axially with respect to the other control handles 220, 320, 420 of the delivery/deployment system 100 to advance the flexible elongate member 1100 and the anchor garage 1200 with respect to the steerable tubular flexible elongate member 310 and to the deployment site. Additionally or alternatively, the delivery and deployment system control handle 1020, operatively associated with the flexible elongate member 1100, may be rotated with respect to the other control handles 220, 320, 420 of the delivery/deployment system 100 to rotate the flexible elongate member 1100 and the anchor garage 1200 with respect to the steerable tubular flexible elongate member 310 and to the deployment site. For instance, in the example of an embodiment illustrated in FIG. 1, the distal end 1021 of the delivery and deployment system control handle 1020 may be supported and/or mounted on a connecting tube 1110 (which may be referenced as a garage telescope shaft for the sake of convenience and without intent to limit) to couple as well as to axially translate (e.g., telescope) and/or rotate the delivery and deployment system control handle 1020 with respect to the other control handles 220, 320, 420 of the delivery/deployment system 100 to axially translate (e.g., telescope) and/or rotate the flexible elongate member 1100 with respect to the steerable tubular flexible elongate member 310 and the treatment site in general. In some embodiments, the distal end 1021 of the deployment system control handle 1020 is fixed with respect to the proximal end 1113 of the connecting tube 1110, and the connecting tube 1110 is slidable and rotatable with respect to the control handle 420 adjacent and distal to the deployment system control handle 1020. For instance, the control handle 420 may include a slider tube therein to telescopically receive the connecting tube 1110 (as shown and described in above-incorporated co-pending provisional patent application ______, [ATTORNEY DOCKET 2001.2754100]. Axial movement of the deployment system control handle 1020 with respect to the control handle 420 telescopes the connecting tube 1110 with respect to the control handle 420 (and, optionally, with respect to a slider tube therein).

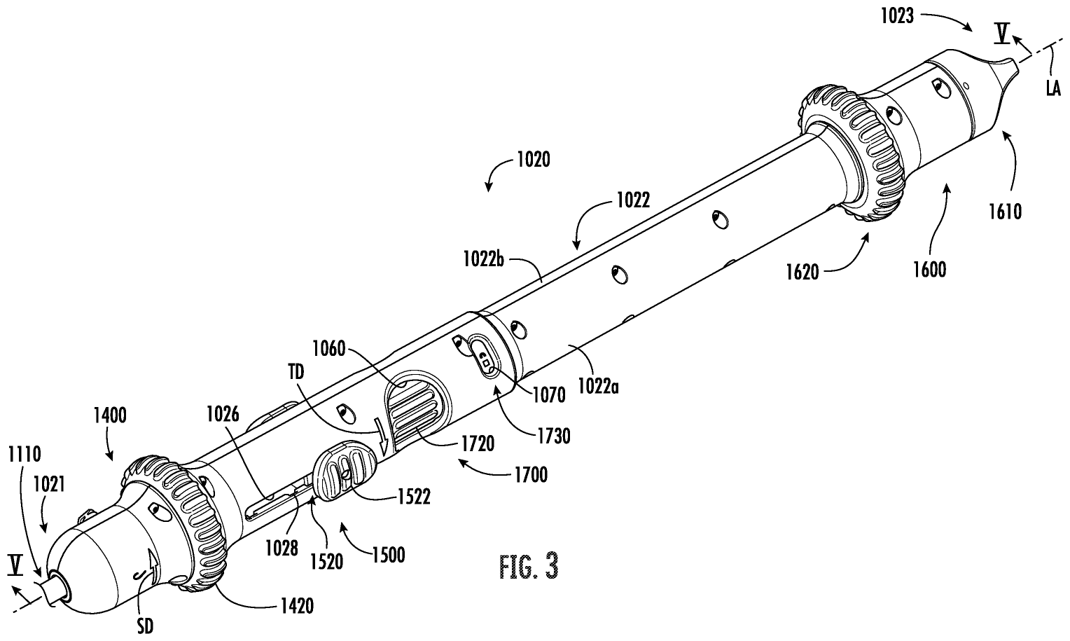

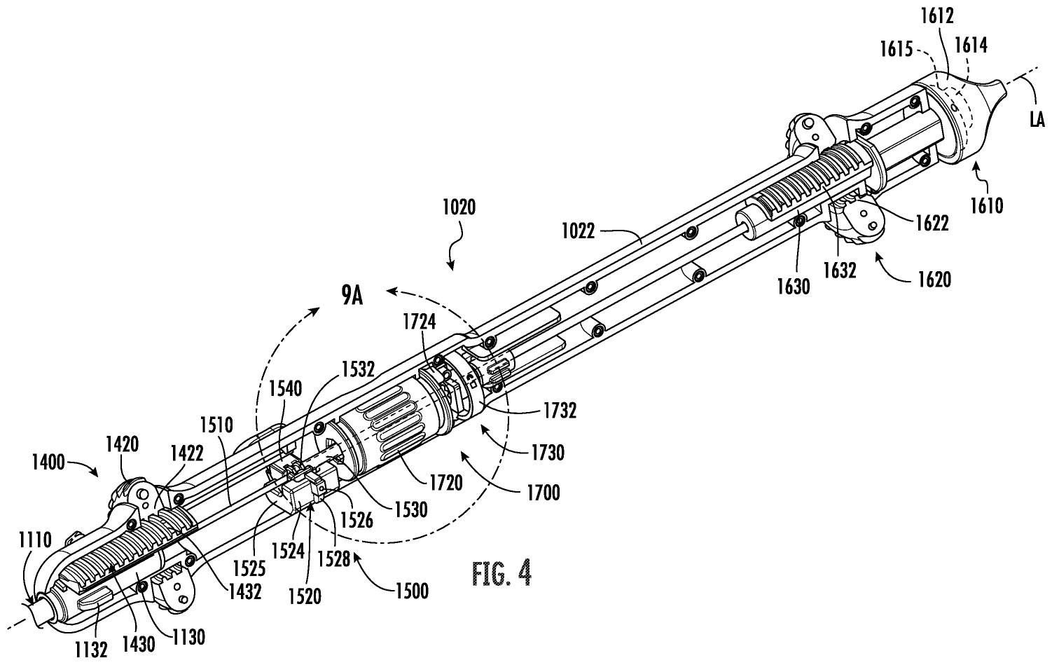

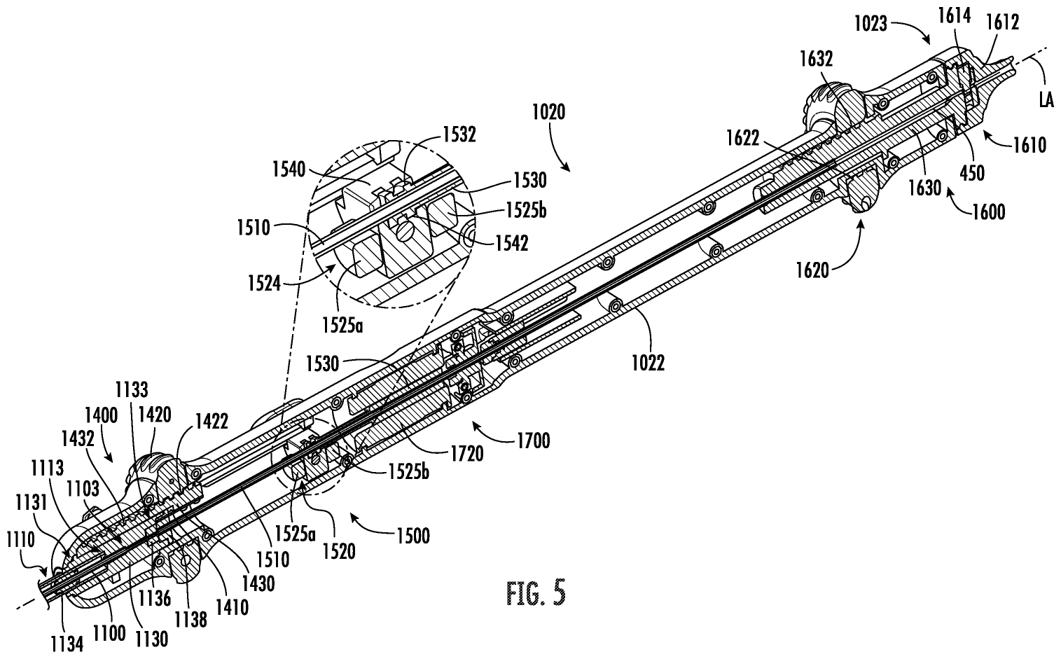

An example of a manner in which the delivery and deployment system control handle 1020 may be mounted with respect to the proximal end 1113 of the connecting tube 1110 and/or the manner in which the flexible elongate member 1100 is mounted with respect to the delivery and deployment system control handle 1020 for movement therewith may be appreciated with reference to FIG. 4 and FIG. 5 (providing interior illustrations of the delivery and deployment system control handle 1020 as illustrated in FIG. 3). In the illustrated example of an embodiment, the proximal end 1113 of the connecting tube 1110 extends into a garage shaft hub 1130 within the steering control knob 1420 and ends in and optionally is bonded within a counterbore within the garage shaft hub 1130 (as illustrated in FIG. 5). The flexible elongate member 1100 extends proximally past the proximal end 1113 of the connecting tube 1110 and the proximal end 1103 of the flexible elongate member 1100 and ends in and optionally is bonded within a counterbore within the garage shaft hub 1130 (as illustrated in FIG. 5), proximal to the counterbore in which the proximal end 1113 of the connecting tube 1110 ends. Optionally, a tubular spacer 1134 guides or supports (and optionally seals) the flexible elongate member 1100 within the connecting tube 1110. With the connecting tube 1110 and the flexible elongate member 1100 bonded with the garage shaft hub 1130, movement of the garage shaft hub 1130 is imparted to the connecting tube 1110 and the flexible elongate member 1100. In turn, the garage shaft hub 1130 is mounted within the delivery and deployment system control handle 1020 so that movement of the delivery and deployment system control handle 1020 is imparted to the connecting tube 1110 and the flexible elongate member 1100. For instance, the garage shaft hub 1130 and the delivery and deployment system control handle 1020 may include keying features that impart movement of the delivery and deployment system control handle 1020 to the garage shaft hub 1130 (and thus the connecting tube 1110 and the flexible elongate member 1100), and which may also mount the garage shaft hub 1130 with respect to the delivery and deployment system control handle 1020. In the example of an embodiment illustrated in FIG. 4, the keying feature includes a wing 1132 extending radially from the garage shaft hub 1130 to engage a corresponding key or keyhole (not shown, but which may be readily understood by those of ordinary skill in the art) within the delivery and deployment system control handle 1020. It will be appreciated that other configurations are within the scope and spirit of the present disclosure, the details of which are not critical to the present disclosure.

Once the desired position of the delivery and deployment system control handle 1020 has been achieved, the delivery and deployment system control handle 1020 may be substantially fixed in the desired position by being fixed with respect to a stand 500 supporting the delivery and deployment system control handle 1020. In the example of an embodiment illustrated in FIG. 1, a thumb screw 520, or the like, may be provided to hold the delivery and deployment system control handle 1020 in place with respect to the stand 500. Further details of the stand are provided and described in above-incorporated co-pending provisional patent application ______, [ATTORNEY DOCKET 2001.2787100].

In the example of an embodiment illustrated in FIG. 2, an example of an embodiment of a deployment system 430 is illustrated in position to deploy a second implantable device with respect to the treatment site. In some embodiments, the steerable tubular flexible elongate member 310 steers the flexible elongate member 410 to the treatment site and the deployment system 430 is axially translatable distally from the steerable tubular flexible elongate member 310 to be deployed/to deploy a second implantable device. For instance, in the example of an anatomical site illustrated in FIG. 2, at which a delivery and deployment system 1000 formed in accordance with various principles of the present disclosure may be used, the deployment system 430 is deployed with respect to a heart valve leaflet L. The flexible elongate member 1100, carrying the anchor garage 1200, is delivered through a lumen within the flexible elongate member 410. As may be appreciated with reference to FIG. 2, once the deployment system 430 has deployed an implantable device (e.g., a leaflet clip 440) with respect to a first anatomical site (e.g., a heart valve leaflet L), the steerable tubular flexible elongate member 310 generally would not be further steered, such as to navigate the flexible elongate member 1100 to a second, different anatomical site (e.g., papillary muscle of the heart). As such, it may be desirable for the flexible elongate member 1100 to have steering capabilities independent of the steerable delivery system 300.

In accordance with various principles of the present disclosure, the flexible elongate member 1100 may include a steering element operatively associated with (e.g., coupled or engaged with to operate) a portion of the wall of the flexible elongate member 1100 along a steerable region of the flexible elongate member 1100 (generally adjacent the distal end 1101 thereof) in any of a variety of manners to effect movement of the steerable region, such as by pulling or otherwise manipulating the steering element. The steering element may be coupled to a portion of the wall of the flexible elongate member 1100 such as by being adhered, bonded, welded, etc., to or within (e.g., embedded within) a portion of the wall of the flexible elongate member 1100. Proximal pulling of the steering element causes the distal end region of the flexible elongate member 1100 (e.g., in the vicinity of the distal end 1101) to bend. It will be appreciated that any of a variety of steering elements known in the art may be used, the present disclosure not being limited to a particular steering element configuration. The steering element may be a generally elongated flexible element which extends generally axially along the flexible elongate member 1100 and the longitudinal axis LA of the delivery and deployment system control handle 1020. In some embodiments, the steering element is a pull mechanism such as a pull element. In some embodiments, the pull mechanism is a two-part mechanism, such as a Bowden cable, with an outer tubular sheath and an inner pull element such as a flexible elongate pull element (e.g., a pull wire). Non-limiting examples of embodiments of steering elements are disclosed in U.S. Patent Application Publication 2023/0011214, titled Radially Clocked Steerable Catheter, and published on Jan. 12, 2023; and U.S. provisional patent application ______, [Attorney Docket 2001.2757100], titled Steerable Member And System And Methods Of Making And Using Same, and filed on Apr. 25, 2022, each of which application is incorporated by reference herein in its entirety and for all purposes. The steering element may be configured to steer the flexible elongate member 1100 in a single plane (e.g., one-way or two-way steering), with rotation of the delivery and deployment system control handle 1020 (such as described above) allowing steering of the flexible elongate member 1100 in different planes. It will be appreciated that because the delivery and deployment system control handle 1020 may be rotatable (e.g., as a unit, including the flexible elongate member 1100), two-way or even one-way steering of the steering element may be sufficient.

In the example of an embodiment of a delivery and deployment system control handle 1020 illustrated in FIG. 1, and in further detail in FIG. 3, FIG. 4, and FIG. 5, a steering system 1400 is provided with a steering control knob 1420 operatively coupled with the flexible elongate member 1100 to control steering of the flexible elongate member 1100. For instance, a steering element 1410 may extend proximally from the flexible elongate member 1100 to be operably engaged with the steering control knob 1420. In the example of an embodiment illustrated in FIG. 5, the steering element 1410 extends proximally from the flexible elongate member 1100 through a gasket 1136 which creates a seal between the interior of the garage shaft hub 1130 and the interior of the delivery and deployment system control handle 1020. In some embodiments, the gasket 1136 has an elongated shape, such as a teardrop shape, to seal around the steering element 1410 as well as around a stylet 1510 which extends proximally out of the proximal end 1103 of the flexible elongate member 1100 (which is operatively coupled at a distal end thereof with the implantable device 1300, as described in further detail below). A cap 1138 may close the proximal end 1133 of the garage shaft hub 1130 with the gasket 1136 therein, and the steering element 1410 and the stylet 1510 extending proximally therethrough. The proximal end of the steering element 1410 may then extend to and be fixed to an axially translatable component 1430 such as by bonding, an interference fit, fixation posts, fixing screws, etc.). Rotation of the steering control knob 1420 about the longitudinal axis LA of the delivery and deployment system control handle 1020 causes generally axial translation of the axially translatable component 1430 along the longitudinal axis LA to pull the steering element 1410 to pull the distal region of the flexible elongate member 1100 proximally to steer the flexible elongate member 1100, such as in a manner as described above. Rotation in the opposite direction release tension on the distal region of the flexible elongate member 1100 (e.g., to allow the distal region of the flexible elongate member 1100 to return to an initial, neutral, straight, unactuated, etc., configuration). The steering control knob 1420 may have a gripping surface, e.g., texturing, knurling, coating, or otherwise, configured to facilitate non-slip gripping thereof to rotate the steering control knob 1420, such as in a manner known to those of ordinary skill in the art. Further details of the example of an embodiment of a steering control system 1400 illustrated in FIG. 1 and FIG. 3 may be appreciated with reference to FIG. 4, and the cross-sectional view along line V-V of FIG. 3 provided by FIG. 5. It is noted that FIG. 4 illustrates a control handle 1020 as in FIG. 3, but with a portion of the housing 1022 thereof removed. For instance, in some embodiments, the delivery and deployment system control handle housing 1022 may be formed from first and second housing halves 1022a, 1022b which are separable, such as to mount the components of the delivery and deployment system control handle 1020 therein.

In accordance with various principles of the present disclosure, in the example of an embodiment of a steering control system 1400 illustrated in FIG. 1, FIG. 3, FIG. 4, and FIG. 5, the steering control knob 1420 is rotatable about the longitudinal axis LA of the delivery and deployment system control handle 1020. Such rotation allows what may be considered intuitive steering of the flexible elongate member 1100 in a steering plane which may correspond generally with the direction in which the steering control knob 1420 is rotated. However, as noted above, the steering element extends generally longitudinally along the flexible elongate member 1100 and the longitudinal axis LA of the delivery and deployment system control handle 1020. Accordingly, rotational movement of the steering control knob 1420 causes axial or longitudinal movement of the axially translatable component 1430 to pull the steering element to bend the flexible elongate member 1100 in the direction in which the steering element is operatively engaged therewith. In some embodiments, retraction of the flexible elongate member 1100 into the flexible elongate member 410 and/or the additional deployment system 430 may facilitate returning the flexible elongate member 1100 to its position prior to actuation by the axially translatable component 1430 such as an initial, neutral, straight, unactuated, etc., position.

In accordance with various principles of the present disclosure, as may be appreciated with reference to FIG. 4 and FIG. 5, the steering control knob 1420 includes internal engagement elements 1422 operatively engaging external engagement elements 1432 on the axially translatable component 1430 to convert rotational movement of the steering control knob 1420 to axial movement of the axially translatable component 1430 to pull the steering element. In some embodiments, the engagement elements 1422, 1432 are threads or components thereof, and the axially translatable component 1430 is a rack gear or slide screw (e.g., partial screw). For instance, the steering control knob 1420 may include internal threads (e.g., at least two or at least three) engaging external thread components (e.g., at least two or at least three) on the axially translatable component 1430. As may readily be appreciated, rotation of the steering control knob 1420 in a steering direction SD (which may be indicated on the delivery and deployment system control handle 1020, along with indicia representing steering/bending of the flexible elongate member 1100, such as illustrated, for example in FIG. 3) causes axial translation of the axially translatable component 1430 in a proximal direction (i.e., toward the proximal end 1023 of the delivery and deployment system control handle 1020) to bend the distal region (adjacent the distal end 1101) of the flexible elongate member 1100. In some embodiments, the flexible elongate member 1100 bends generally in a direction corresponding to the direction in which the steering control knob 1420 is turned, such as may be appreciated with reference to FIG. 1.

Once the delivery and deployment system 1000 delivers the flexible elongate member 1100 to the treatment site, such as with the use of the steering control system 1400, the implantable device 1300 may be deployed. For instance, in the example of an embodiment illustrated in FIGS. 1-5, the implantable device 1300 is delivered to the treatment site within an anchor garage 1200 at the distal end 1101 of the flexible elongate member 1100, such as described above. Once the anchor garage 1200 is at the treatment site, the implantable device 1300 may be deployed therefrom.

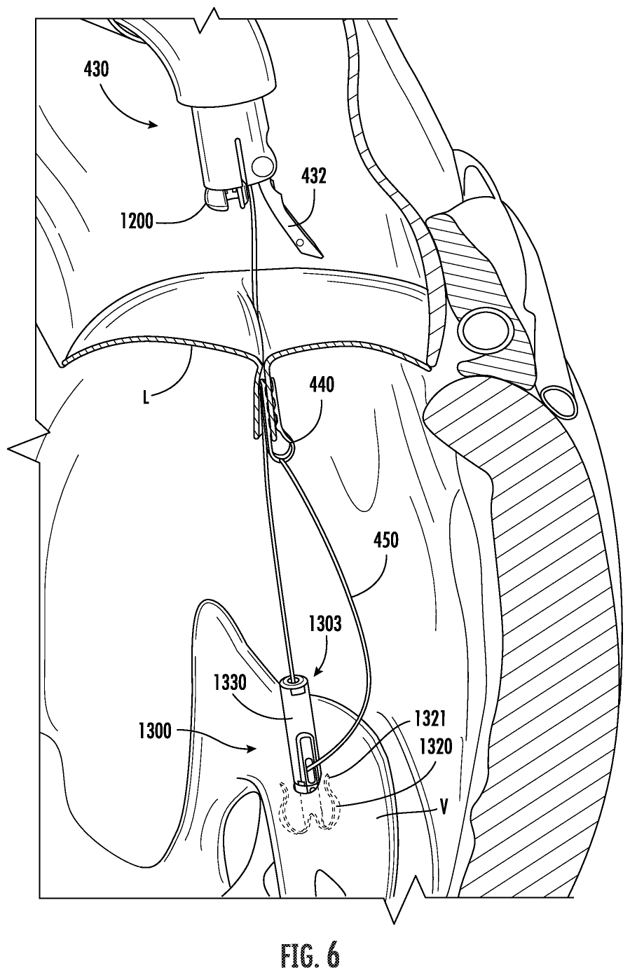

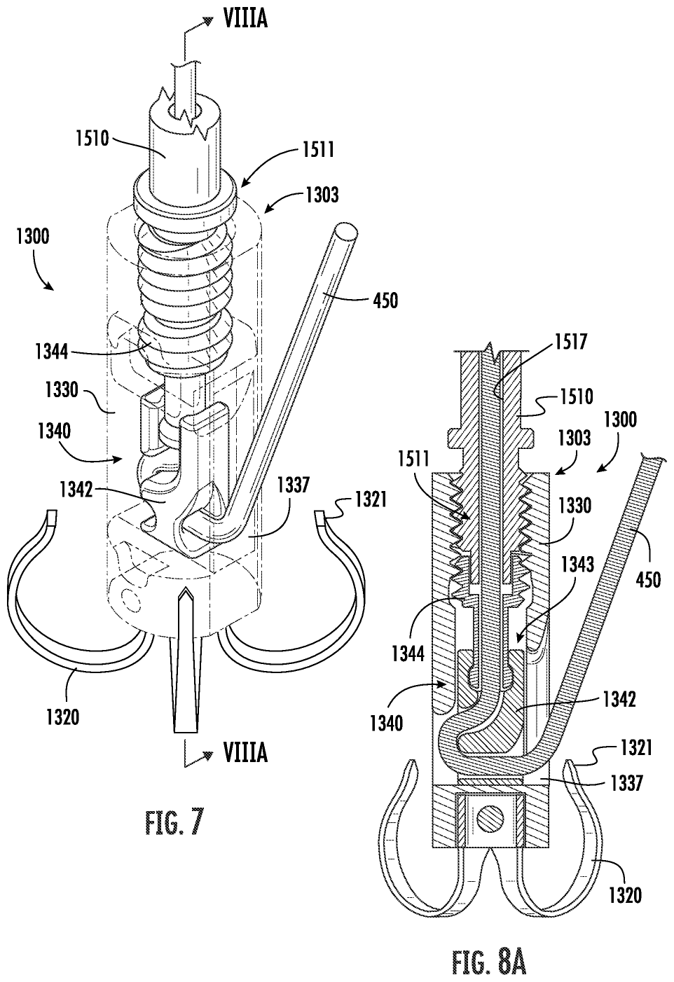

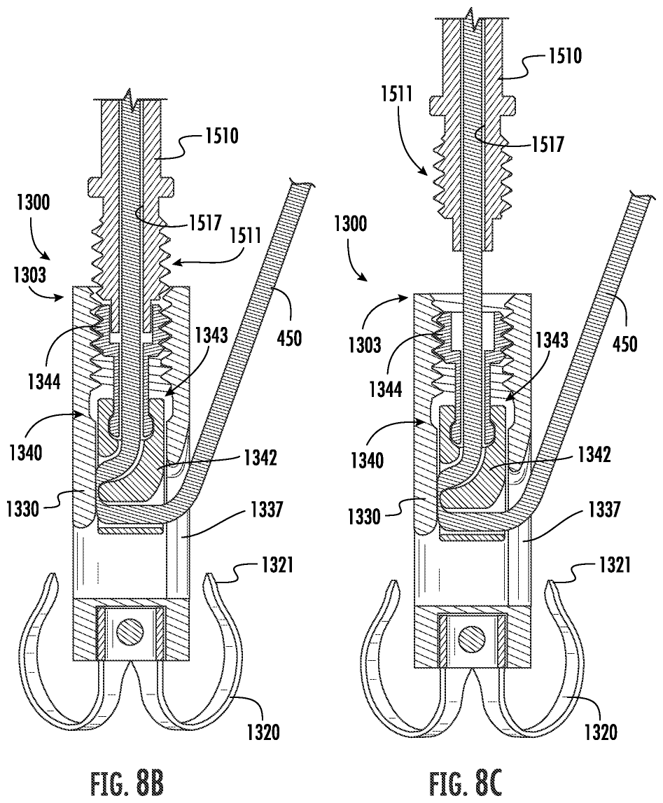

The example of an embodiment of a delivery and deployment system 1000 illustrated in FIGS. 1-5 includes an implantable device deployment system 1500 (referenced herein as an anchor deployment system 1500 for the sake of convenience and without intent to limit) configured to deploy an implantable device 1300 from the implantable device delivery device 1200. In the illustrated example of an embodiment, the anchor deployment system 1500 includes a stylet 1510 (illustrated in phantom in FIG. 2) operatively engaged with the implantable device 1300 to deliver and deploy the implantable device 1300. For instance, the implantable device 1300 may be coupled to the distal end 1511 of the stylet 1510, as illustrated in FIG. 7, and FIGS. 8A-8D. In the example of an embodiment illustrated in FIG. 2, the implantable device 1300 is illustrated as a tissue anchor, and reference is thus made herein to either an implantable device 1300 or an anchor 1300 interchangeably and without intent to limit. As may be appreciated with reference to FIG. 2, the anchor 1300 is delivered in the anchor garage 1200 in a delivery configuration in which the anchor talons 1320 are extended distally and the anchor 1300 is thus in a relatively compact configuration. When deployed distally out of the anchor garage 1200, the anchor 1300 shifts into an expanded deployed configuration, such as illustrated in FIG. 6, FIG. 7, and FIG. 8A-8D. In the illustrated example of an embodiment of an anchor 1300, in the deployed configuration, the anchor talons 1320 curl back proximally (e.g., towards the proximal end 1303 of the anchor 1300) into an expanded, optionally bowed configuration. The anchor talons 1320 may be formed of a shape-memory material (e.g., nitinol) to shift from the delivery configuration to the deployed configuration upon exiting the anchor garage 1200 and without further forces being applied thereto. As such, as the anchor 1300 is advanced out of the anchor garage 1200, it is important that the distal ends 1321 of the anchor talons 1320 pierce the tissue into which the anchor 1300 is to be engaged so that the anchor talons 1320 extend into the deployed configuration within the tissue (and not upon extending out of the anchor garage 1200 and before being advanced into the tissue). The distal ends 1321 may be shaped (e.g., sharp, pointed, etc.) to facilitate piercing and penetrating into tissue.

In accordance with various principles of the present disclosure, the anchor deployment system 1500 is configured to control deployment of the anchor 1300. In the example of an embodiment illustrated in FIG. 1, FIG. 3, FIG. 4, and FIG. 5, actuation of the anchor deployment system 1500 axially translates the anchor 1300 with respect to the anchor garage 1200 to advance the anchor 1300 with respect to the anchor garage 1200 (e.g., to deploy the anchor 1300), and/or retract the anchor 1300 with respect to the anchor garage 1200 (e.g., to withdraw within the anchor garage 1200). Advancement of the anchor 1300 out of the anchor garage 1200 deploys the anchor 1300, such as into tissue. If desired, generally before deployment, the anchor 1300 may be recaptured within the anchor garage 1200 by retracting the anchor 1300, such as with the use of the anchor deployment system 1500.

The example of an embodiment of an anchor deployment system 1500 illustrated FIG. 1, FIG. 3, FIG. 4, and FIG. 5 includes an anchor control slider 1520 operable to advance or retract (e.g., axially translate) the stylet 1510 with respect to the delivery and deployment system control handle 1020, the flexible elongate member 1100, and the anchor garage 1200 to advance or retract the anchor 1300 with respect to the anchor garage 1200. More particularly, as illustrated FIG. 4 and FIG. 5, the stylet 1510 of the illustrated example of an embodiment of an anchor deployment system 1500 extends from within the flexible elongate member 1100 proximally to be mounted (e.g., fixedly, such as with mating engagements, adhesive, bonding, etc.) within a stylet hub 1530. As noted above, a gasket 1136 may be provided within the proximal end 1133 of the garage shaft hub 1130 to seal any fluids from within the flexible elongate member 1100 from extending proximally into the interior of the delivery and deployment system control handle 1020 by forming a seal around the stylet 1510 and between the interior of the garage shaft hub 1130 (including the counterbore in which the proximal end 1103 of the flexible elongate member 1100 is seated) and the interior of the delivery and deployment system control handle 1020. The stylet hub 1530 is configured to be operably engaged with a carriage 1540 operably coupled with the anchor control slider 1520 to axially translate therewith. For instance, the stylet hub 1530 and the carriage 1540 may include matingly engaging one or more (e.g., two or more or three or more, etc.) projections 1532 and one or more (e.g., two or more or three or more, etc.) corresponding recesses 1542. It will be appreciated that although the projections 1532 are illustrated on the stylet hub 1530, and the recesses 1542 are illustrated on the carriage 1540, the reverse arrangement is within the scope and spirit of the present disclosure as well. As discussed in further detail below, the stylet hub 1530 and the carriage 1540 preferably are rotatably engaged with respect to each other to allow rotation of the stylet 1510 with respect to the delivery and deployment system control handle housing 1022. The carriage 1540 is matingly engaged with an anchor control slider base 1524 having a projection 1526 extending through a slot 1026 in the delivery and deployment system control handle housing 1022 (see, e.g., FIG. 3) and on which the slider button 1522 is mounted. For instance, the anchor control slider base 1524 may include legs 1525a, 1525b extending on either side of the carriage 1540 so that axial translation or sliding of the slider button 1522 and the anchor control slider base 1524 along the longitudinal axis LA of the delivery and deployment system control handle 1020 is imparted to the carriage 1540. It will be appreciated that a reverse arrangement, with the carriage 1540 having legs on either side of the anchor control slider base 1524 is within the scope and spirit of the present disclosure as well. As such, a user may engage the slider button 1522 to slide the anchor control slider base 1524 and thus the carriage 1540 and the stylet hub 1530 with respect to the delivery and deployment system control handle 1020 to extend or retract the anchor 1300 from or into the anchor garage 1200. Optionally, the slider button 1522 has a gripping surface (e.g., textured, coating, grooved or otherwise to facilitate non-slip engagement thereof) to facilitate engagement of the slider button 1522 by a user to enhance grip/reduce slipping of the user's finger with respect to the slider button 1522.

As discussed above, distal translation of the slider button 1522 with respect to the delivery and deployment system control handle housing 1022 may deploy the anchor 1300 from the anchor garage 1200. Generally, it is desirable to prevent premature extension of the anchor 1300 from the anchor garage 1200, particularly if the anchor 1300 shifts into a deployed configuration upon exiting the anchor garage 1200 even before contacting a desired deployment site. In accordance with various principles of the present disclosure, the anchor control slider 1520 may be locked in one or more positions with respect to the delivery and deployment system control handle housing 1022. For instance, the anchor control slider base 1524 may include a boss 1528 engageable with notches 1028 in the delivery and deployment system control handle housing 1022, as may be appreciated with reference to FIG. 3 and FIG. 4. It will be appreciated that other configurations and structures are within the scope and spirit of the present disclosure as well. The slider button 1522 may be locked with respect to the delivery and deployment system control handle 1020 to prevent inadvertent advancement of the anchor 1300 from the anchor garage 1200, which may cause inadvertent deployment of the anchor 1300. In some embodiments, the slider button 1522 is biased with respect to the carriage 1540 to lock the boss 1528 thereon into a corresponding notch 1028 in the delivery and deployment system control handle housing 1022. For instance, a biasing element such as a coil spring may be provided between the slider button 1522 and the carriage 1540. Pressing of the slider button 1522 towards the carriage 1540 and against the biasing force of the biasing element releases the boss 1528 from the notch 1028 to allow selective movement of the anchor control slider 1520 to translate the stylet 1510 and thus the anchor 1300 as desired. As described above, as the flexible elongate member 1100 is navigated through passages within a patient's body, compressive forces may act upon the flexible elongate member 1100. To counteract such forces, which may result in retracting the anchor garage 1200 with respect to the anchor 1300 therein and inadvertently prematurely deploying the anchor 1300, tension may be applied to the stylet 1510 to maintain the anchor 1300 within the anchor garage 1200. For instance, as the flexible elongate member 1100 and the stylet 1510 are distally advanced transluminally through a patient's body, the anchor control slider 1520 may be retracted incrementally as needed to apply tension to the stylet 1510 to maintain the anchor 1300 withdrawn within the anchor garage 1200.

In the example of an anatomical site illustrated in FIG. 2 to which the flexible elongate member 1100 may be navigated, the delivery and deployment system 1000 delivers an implantable device 1300 in the form of a tissue anchor 1300 to cardiac tissue to be implanted therein. As noted above, the delivery/deployment system 100 may deliver, operate, and/or deploy cardiac repair devices and/or systems, such as heart valve leaflet repair devices and/or systems. For instance, as described above, the delivery/deployment system 100 may include an additional delivery and deployment system 400 which may deliver a deployment system 430 to a deployment site to deploy an additional implantable device 440. In the example of an embodiment illustrated in FIG. 2 and FIG. 6, the deployment system 430 is a leaflet clip spreader configured to deploy an additional implantable device 440 in the form of a leaflet clip. The deployment system 430 thus may be alternately referenced herein as a leaflet clip spreader 430, and the additional implantable device 440 may be alternately referenced herein as a leaflet clip 440 without intent to limit. The leaflet clip 440 and the anchor 1300 may be used in conjunction with each other to implant a tether element 450, such as a suture or an artificial chordae tendineae 450 (reference being made to such elements interchangeably herein and without intent to limit), with respect to a heart valve leaflet L and heart ventricle V, such as illustrated in FIG. 6, to repair the function of the heart valve leaflet L. The leaflet clip 440 may be deployed by the leaflet clip spreader 430, on a heart valve leaflet L, with the artificial chordae tendineae 450 coupled thereto and extending to the anchor 1300 within the anchor garage 1200.

As may be appreciated, it may be desirable to adjust the tension on the artificial chordae tendineae 450. In accordance with various principles of the present disclosure, the artificial chordae tendineae 450 is movably coupled with respect to the anchor 1300. For instance, in the example of an embodiment illustrated in FIG. 2 and FIG. 6, and in greater detail in FIG. 7, FIG. 8A, FIG. 8B, FIG. 8C, and FIG. 8D, the artificial chordae tendineae 450 may movably extend through an anchor housing 1330 of the anchor 1300 (from which the anchor talons 1320 extend distally). In particular, the artificial chordae tendineae 450 may extend through a lumen 1517 through the stylet 1510 and into operative engagement with the anchor 1300. However, other entrance points for the artificial chordae tendineae 450 are within the scope and spirit of the present disclosure. The amount of tension on and/or length of the artificial chordae tendineae 450 extending between the leaflet clip 440 and the anchor 1300 may be adjusted by pulling or paying out the artificial chordae tendineae 450 with respect to the anchor 1300. For instance, the artificial chordae tendineae 450 may extend through the proximal end 1023 of the delivery and deployment system control handle 1020 and may be manually manipulated by the medical professional operating the delivery and deployment system 1000, such as by directly manipulating the artificial chordae tendineae 450 by hand (e.g., grasping and pulling on the artificial chordae tendineae 450). However, it will be appreciated that such adjustment may be considered a gross adjustment, and fine adjustment generally is desirable to achieve the proper heart valve leaflet functioning to be achieved by implanting the leaflet clip 440, anchor 1300, and artificial chordae tendineae 450.

In accordance with various principles of the present disclosure, the example of an embodiment of a delivery and deployment system control handle 1020 illustrated in FIG. 1, FIG. 3, FIG. 4, and FIG. 5 includes a tether adjustment system 1600 configured to engage and adjust tension on and/or the length of the tether elements. The illustrated example of an embodiment of a tether adjustment system 1600, alternately referenced herein as a suture adjustment system 1600, includes a suture tail lock 1610 and a suture fine tuning knob 1620 mounted adjacent/along the proximal end 1023 of the delivery and deployment system control handle 1020 for gross and fine control of the length of/tension on the tether element 450, alternately referenced herein as a suture 450. As may be further appreciated with reference to FIG. 5, the suture tail lock 1610 includes a suture locking knob 1612 and a suture lock cam 1614. The suture 450 extends axially through the suture lock cam 1614, and the suture lock cam 1614 is shiftable with respect to another element, such as a suture tension lead screw 1630, between a position in which the suture 450 freely moves through the suture lock cam 1614 and a position in which the suture 450 is locked against movement. More particularly, the suture lock cam 1614 is shiftable to pinch or wedge the suture 450 between the suture lock cam 1614 and the suture tension lead screw 1630 on which the suture lock cam 1614 is mounted. As illustrated example in the detail view of FIG. 4, the suture locking knob 1612 has a cam surface 1615 engaging the suture lock cam 1614 so that upon rotation of the suture locking knob 1612 about the longitudinal axis LA of the delivery and deployment system control handle 1020, the suture lock cam 1614 shifts transverse to the longitudinal axis LA into and out of a position locking the suture 450 in place with respect to the suture tension lead screw 1630. The medical professional may adjust the tension and/or length of the artificial chordae tendineae 450 between the leaflet clip 440 and the anchor 1300, with the suture locking knob 1612 in an unlocked position, such as by using appropriate imaging techniques to observe the effect of the tension on the heart valve being repaired (e.g., observing the blood flow therethrough, proper tension on the heart valve eliminating any regurgitation therethrough). Once the desired heart valve function is observed and confirmed, for instance, once regurgitative blood flow has been eliminated or at least sufficiently reduced, the suture locking knob 1612 may be rotated to shift the suture lock cam 1614 into a locking position, thereby locking the suture adjustment system 1600 and setting the tension on/length of the artificial chordae tendineae 450.

Once the general tension on/length of the artificial chordae tendineae 450 has been adjusted, fine adjustments may still be medically indicated to fine tune the heart valve functioning. In accordance with various further principles of the present disclosure, the suture adjustment system 1600 also includes a suture fine tuning knob 1620 which may then be actuated to further adjust the tension on/length of the artificial chordae tendineae 450 in finer/smaller increments than achieved when simply adjusting the tension/length manually by the medical professional grasping the artificial chordae tendineae 450 by hand. The example of an embodiment of a suture fine tuning knob 1620 illustrated in FIG. 1, FIG. 3, FIG. 4, and FIG. 5 is rotatable about the longitudinal axis LA of the delivery and deployment system control handle 1020 to axially advance or retract the suture tension lead screw 1630. With the artificial chordae tendineae 450 substantially fixed with respect to the suture tension lead screw 1630 by the suture lock cam 1614 in a locked position, axial translation of the suture tension lead screw 1630 causes corresponding axial translation of the artificial chordae tendineae 450 to adjust the tension on/length of the artificial chordae tendineae 450. Movement of the suture fine tuning knob 1620 may be translated to movement of the suture tension lead screw 1630 in any of a variety of manners. In the example of an embodiment illustrated in FIG. 4 and FIG. 5, rotational movement of the suture fine tuning knob 1620 may cause axial translation of the suture tension lead screw 1630 in a manner similar to that described with respect to the steering control knob 1420 and the axially translatable component 1430. For instance, the suture fine tuning knob 1620 may include internal engagement elements 1622 operatively engaging external engagement elements 1632 on the suture tension lead screw 1630 to convert rotational movement of the suture fine tuning knob 1620 to axial movement of the suture tension lead screw 1630 to pull the steering element. In some embodiments, the engagement elements 1622, 1632 are threads or components thereof. For instance, the suture tension lead screw 1630 may be a rack gear or slide screw (e.g., partial screw) with external thread components (e.g., at least two or at least three) operatively engaged with (e.g., at least two or at least three) internal threads provided on the suture fine tuning knob 1620. As may readily be appreciated, rotation of the suture fine tuning knob 1620 causes axial translation of the suture tension lead screw 1630 to adjust the tension on/length of the artificial chordae tendineae 450.

Once the desired tension on and/or length of the artificial chordae tendineae 450 has been attained with the suture fine tuning knob 1620, it may be desirable to lock the artificial chordae tendineae 450 against further adjustment with respect to the anchor 1300. In an example of an embodiment of an anchor 1300 illustrated in FIG. 7, a tensioning and locking device 1340 may be provided within the anchor housing 1330 to lock the artificial chordae tendineae 450 with respect to the anchor housing 1330. In accordance with various principles of the present disclosure, the stylet 1510 is operably engaged with the tensioning and locking device 1340 to move the tensioning and locking device 1340 with respect to the anchor housing 1330, such as to shift between an tension-adjusting configuration in which the artificial chordae tendineae 450 is adjustable, such as illustrated in FIG. 8A, and a tension-setting configuration in which the tension on and/or length of the artificial chordae tendineae 450 is set/fixed/locked (such terms being used interchangeably herein without intent to limit), such as illustrated in FIG. 8B. Once the tension on/length of the artificial chordae tendineae 450 is set, the stylet 1510 may be disengaged from the anchor 1300, such as illustrated in FIG. 8C.

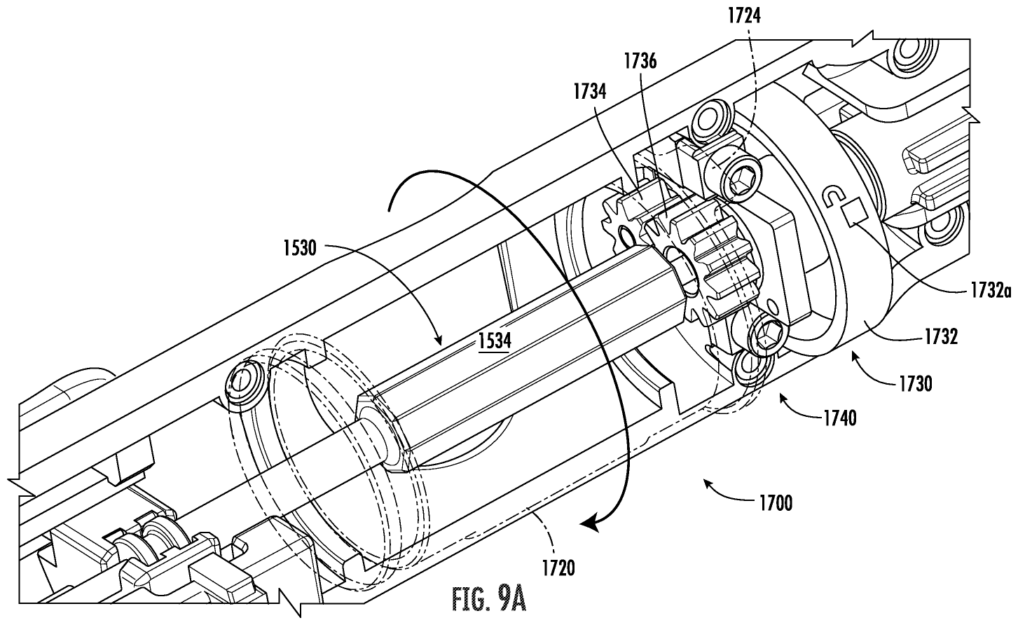

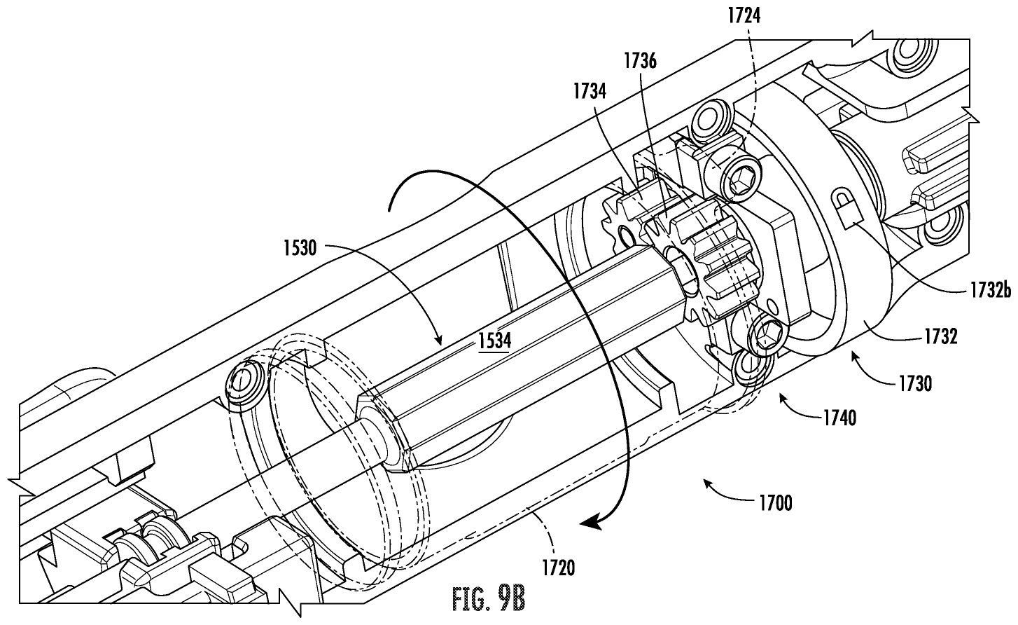

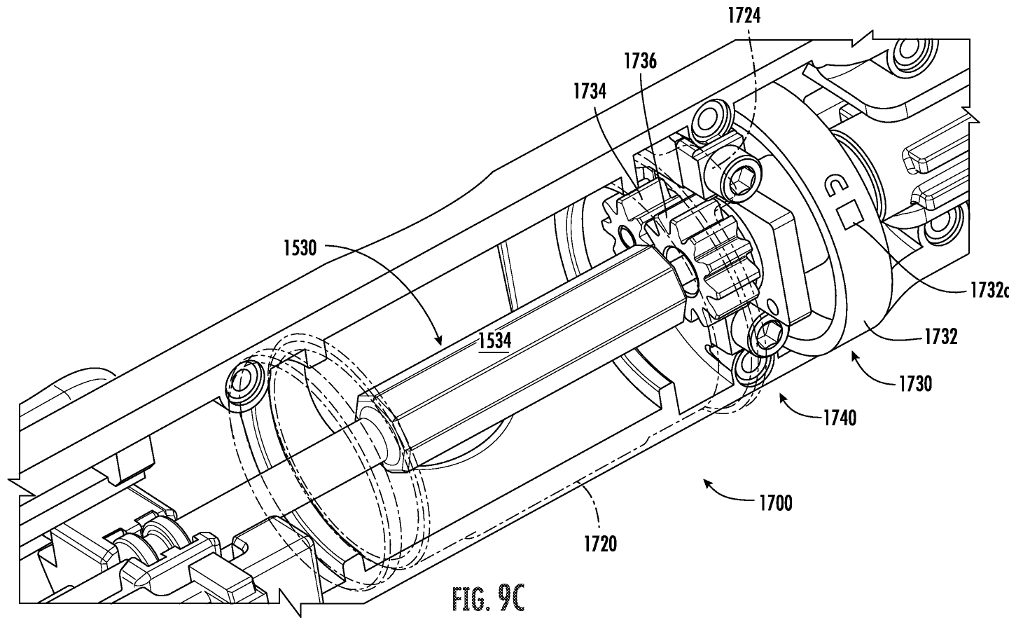

More particularly, the example of an embodiment of a delivery and deployment system 1000 illustrated in FIG. 1, FIG. 3, FIG. 4, FIG. 5, FIG. 9A, FIG. 9B, and FIG. 9C, includes a tensioning and locking system 1700 having a tensioning and locking knob 1720 operably engaged with the stylet 1510, such as with the stylet hub 1530, to actuate the stylet 1510. In the illustrated example of an embodiment, the tensioning and locking knob 1720 is positioned within the delivery and deployment system control handle housing 1022 to be rotatable about the longitudinal axis LA of the delivery and deployment system control handle 1020. An external surface of the tensioning and locking knob 1720 is accessible through a window 1070 in the delivery and deployment system control handle housing 1022 and may have a gripping surface, e.g., texturing, knurling, coating, or otherwise, configured to facilitate non-slip gripping thereof to rotate the steering control knob 1420, such as in a manner known to those of ordinary skill in the art. The stylet 1510 may be operably engaged with the tensioning and locking knob 1720 to rotate therewith about the longitudinal axis LA of the delivery and deployment system control handle 1020, yet remain free to axially translate with respect to the tensioning and locking knob 1720 along the longitudinal axis LA. For instance, in the example of an embodiment illustrated in FIG. 9A, FIG. 9B, and FIG. 9C, the exterior of the stylet hub 1530 includes one or more flats 1534 (flat surfaces) engaging respective flats within the tensioning and locking knob 1720. As such, rotation of the tensioning and locking knob 1720 imparts rotational movement to the stylet hub 1530 and the stylet 1510. However, the stylet hub 1530 remains free to move axially with respect to the tensioning and locking knob 1720, such as to be advanced or retracted (e.g., with the anchor control slider 1520, as described above, or to adjust the tensioning and locking device 1340, as described below).

Adjustment of the tensioning and locking knob 1720 actuates the stylet 1510 to shift the tensioning and locking device 1340 between a tension-adjusting configuration and a tension-setting configuration, and optionally also to disengage the stylet 1510 from the anchor 1300 (such as to deploy the anchor 1300). For instance, in the example of an embodiment of a tensioning and locking device 1340 illustrated in FIG. 7, FIG. 8A, FIG. 8B, and FIG. 8C, the stylet 1510 is operably coupled with a tensioning and locking device carriage 1342 via a coupler 1344 to axially translate the tensioning and locking device carriage 1342 between a tension-adjusting configuration (FIG. 8A) and a tension-setting configuration (FIG. 8B). For example, the stylet 1510 may be operatively engaged with the coupler 1344 to impart rotational movement thereto. Additionally, the stylet 1510 (e.g., the distal end 1511 thereof) and the coupler 1344 may be threadedly engaged within the anchor housing 1330 such that rotation thereof also causes axial translation thereof with respect to the anchor housing 1330. The coupler 1344 may be operably coupled with respect to the tensioning and locking device carriage 1342 to rotate with respect thereto so that rotation of the coupler 1344 (such as caused by rotation of the stylet 1510) does not cause rotation of the tensioning and locking device carriage 1342, but axial translation of the coupler 1344 does cause axial translation of the tensioning and locking device carriage 1342. As such, rotation of the tensioning and locking knob 1720 causes rotation of the stylet 1510 and the coupler 1344 to cause axial translation of the stylet 1510, the coupler 1344, and the tensioning and locking device carriage 1342 with respect to the anchor housing 1330.

In the example of an embodiment illustrated in FIG. 7, FIG. 8A, FIG. 8B, and FIG. 8C, the artificial chordae tendineae 450 extends generally from a proximal direction (e.g., from the delivery and deployment system control handle 1020) through or along the stylet 1510, generally axially through a proximal end 1343 of the tensioning and locking device carriage 1342, and transversely out the tensioning and locking device carriage 1342 and the anchor housing 1330 (e.g., through a window 1337 in the anchor housing 1330). The artificial chordae tendineae 450 may then extend to another implantable device, such as a leaflet clip 440, such as illustrated in FIG. 6. In the tension-adjusting configuration, such as illustrated in FIG. 8A, the artificial chordae tendineae 450 is freely movable with respect to the tensioning and locking device carriage 1342. In such configuration, tension on and/or the length of the artificial chordae tendineae 450 may be adjusted in any of the manners described above. Rotation of the tensioning and locking knob 1720 proximally advances the tensioning and locking device carriage 1342 with respect to the anchor housing 1330 to the tension-setting configuration illustrated in FIG. 8B. In the illustrated tension-setting configuration, the artificial chordae tendineae 450 is locked against movement by being caught or held in place (e.g., pinched) between the tensioning and locking device carriage 1342 and the interior of the anchor housing 1330. Further rotation of the tensioning and locking knob 1720 decouples the stylet 1510 from the coupler 1344, such as illustrated in FIG. 8C, such as to deploy the anchor 1300. Any of a variety of tensioning and locking devices may be used in connection with the delivery and deployment system 1000 of the present disclosure, such as, without limitation, any of the tensioning and locking devices or systems described in U.S. Patent Application Publication US2022/0096235, published on Mar. 31, 2022, and titled Devices, Systems, And Methods For Adjustably Tensioning Artificial Chordae Tendineae In A Heart, the entirety of which application is incorporated by reference herein in its entirety and for all purposes. It will be appreciated that the present disclosure need not be limited by the structure, form, function, etc., of the tensioning and locking device 1340 illustrated and described in such patent or herein.

In accordance with various principles of the present disclosure, the tensioning and locking system 1700 includes an anchor state indicator 1730 operably associated with the tensioning and locking knob 1720 to indicate the state or configuration of the tensioning and locking device 1340 of the anchor 1300. For instance, as illustrated in FIG. 9A, FIG. 9B, and FIG. 9C, the anchor state indicator 1730 may include an anchor state dial 1732 with indicia 1732A, 1732B, and 1732C representing the state of operation or configuration of the tensioning and locking device 1340. The indicia 1732A, 1732B, and 1732C may be viewable through a window 1070 in the delivery and deployment system control handle housing 1022, such as illustrated in FIG. 3. More particularly, when the tensioning and locking device 1340 is in a tension-adjusting configuration, such as illustrated in FIG. 8A, the anchor state dial 1732 may be positioned with a corresponding “unlocked” indicium 1732A, such as illustrated in FIG. 9A, viewable through the window 1070. When the tensioning and locking device 1340 is in a tension-setting configuration, such as illustrated in FIG. 8B, the anchor state dial 1732 may be positioned with a corresponding “locked” indicium 1732B, such as illustrated in FIG. 9B, viewable through the window 1070. Finally, when the stylet 1510 has been decoupled from the anchor 1300, such as illustrated in FIG. 8C, the anchor state dial 1732 may be positioned with a corresponding “detached” or “dissociated” indicium 1732C, such as illustrated in FIG. 9C, viewable through the window 1070.

The tensioning and locking knob 1720 may be operatively coupled with the anchor state dial 1732 in any of a variety of manners known to those of ordinary skill in the art. For instance, in the example of an embodiment illustrated in FIGS. 9A-C, rotation of the tensioning and locking knob 1720 may be transferred to rotation of the anchor state dial 1732 via a gear coupling 1740. More particularly, the illustrated example of an embodiment of a gear coupling 1740 includes an actuator spur gear 1734 engageable by an actuator finger 1724 extending axially and proximally from the tensioning and locking knob 1720. The tensioning and locking knob 1720 may be turned in a tensioning direction TD (which may be indicated on the delivery and deployment system control handle 1020, such as illustrated, for example in FIG. 3) to shift the tensioning and locking device 1340 from the tension-adjusting configuration to the tension-setting configuration. Upon each full rotation (360 degrees) of the tensioning and locking knob 1720, the actuator finger 1724 causes an incremental rotation of the actuator spur gear 1734. The actuator spur gear 1734, in turn, causes an incremental rotation of a dial spur gear 1736 which rotates with the anchor state dial 1732. The gear rations of the spur gears 1734 and 1736 may be chosen based on the diameter of the anchor state dial 1732, the diameter of the stylet 1510 and/or stylet hub 1530, the length of the tensioning and locking device 1340 and/or tensioning and locking device carriage 1342 and/or coupler 1344, etc., to correlate the configuration of the tensioning and locking device 1340 with the anchor state dial 1732, as may appreciated by one of ordinary skill in the art.

As may be appreciated by one of ordinary skill in the art, a delivery and deployment system 1000 and a delivery and deployment system control handle 1020 as described herein has a variety of structures and uses and functions which may be separate and independent of one another, and/or may operate in conjunction with one another. The delivery and deployment system control handle 1020 described herein may be used in conjunction with a delivery system 200, and/or a steerable delivery system 300, and/or an additional device delivery and deployment system 400 of a delivery/deployment system 100 such as described above. For instance, in some embodiments, an implantable device and an additional implantable device may be delivered to a heart valve to perform a cardiac repair procedure with the use of a delivery system 200, a steerable delivery system 300, and an additional device delivery and deployment system 400, such as described above. The cardiac repair procedure may be a heart valve repair procedure in which a leaflet clip 440 is coupled to a heart valve with an artificial chordae tendineae 450 coupled thereto. In such embodiment, the additional device delivery and deployment system 400 may be delivered and steered into an appropriate position to deliver and deploy a leaflet clip 440, such as by clamping the leaflet clip 440 onto a heart valve leaflet L. The steering control system 1400 may then be operated, such as described above, to steer the anchor garage 1200 to an appropriate position (e.g., with respect to papillary muscle) to anchor the artificial chordae tendineae 450 to the ventricle V. The anchor deployment system 1500 may then be used to advance the anchor 1300 into heart tissue to anchor the artificial chordae tendineae 450 with respect to the ventricle V. Optionally, the leaflet clip 440 is deployed and the deployment system 430 is withdrawn from the ventricle so that proper placement of the leaflet clip 440 and the anchor 1300 may be confirmed, and tension on/the length of the artificial chordae tendineae 450 may be adjusted to result in proper functioning of the heart valve leaflet L and heart valve. The suture adjustment system 1600 may be used such as described above to adjust the tension on/the length of the artificial chordae tendineae 450. Once healthy functioning of the heart valve leaflet L has been achieved, the tensioning and locking system 1700 may be used such as described above to set/lock the tension on/length of the artificial chordae tendineae 450. The stylet 1510 which is coupled to the anchor 1300 to assist with delivering and deploying the anchor 1300 may then be decoupled from the anchor 1300 and withdrawn from the heart. The remaining artificial chordae tendineae 450 proximally extending from the artificial chordae tendineae 450 to the medical professional (e.g., for adjustment) may be cut at this time at a location appropriately close to the anchor 1300 so that excess loose artificial chordae tendineae 450 is not left dangling within the heart.

Although embodiments of the present disclosure may be described with specific reference to an implant for use with mitral valves, it is appreciated that various other implants may similarly benefit from the structures and manufacturing methods disclosed herein. In view of the above, it should be understood that the various embodiments illustrated in the figures have several separate and independent features and methods of use, which each, at least alone, has unique benefits which are desirable for, yet not critical to, the presently disclosed devices, systems, and methods. Therefore, the various separate features, devices, systems, methods, etc., described herein need not all be present in order to achieve at least some of the desired characteristics and/or benefits described herein. Only one of the various features, devices, systems, methods, etc., may be present in in accordance with various principles of the present disclosure. Alternatively, one or more of the features, devices, systems, methods, etc., may be used in various combinations with one another. Various further benefits of the various aspects, features, components, and structures of devices, systems, methods, etc. such as described above, in addition to those discussed above, may be appreciated by those of ordinary skill in the art.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A delivery and deployment system for delivering and/or deploying an implantable device to an anatomical site, said delivery and deployment system comprising:
a flexible elongate member;
an implantable device delivery device deliverable at a distal end of the flexible elongate member to an anatomical site;
an implantable device deliverable by said implantable device delivery device; and
a control handle comprising one or more of the following:
a steering system operably coupled with said flexible elongate member to control movement of said flexible elongate member to deliver said implantable device to the anatomical site;
an implantable device deployment system operably coupled with said implantable device to deploy said implantable device at the anatomical site;
a tether adjustment system operably engageable with a tether element engageable with said implantable device; or
a tensioning and locking system operably engageable with the implantable device to adjust a configuration of a tensioning and locking device operably associated with said implantable device.

2. The delivery and deployment system of claim 1, wherein:
said flexible elongate member includes a pull element operatively associated therewith; and
said control handle comprises at least a steering system, said steering system comprising a steering control knob rotatable about the longitudinal axis of said control handle to pull said pull element axially to steer said flexible elongate member.

3. The delivery and deployment system of claim 1, wherein said implantable device is a tissue anchor.

4. The delivery and deployment system of claim 3, wherein said flexible elongate member is tubular, said delivery and deployment system further comprising a stylet operably coupled with said implantable device and extending through said tubular flexible elongate member.

5. The delivery and deployment system of claim 4, wherein said control handle comprises at least an implantable device deployment system operably coupled with said stylet to axially translate said stylet along the longitudinal axis of said control handle to advance and to deploy the implantable device from the implantable device delivery device.

6. The delivery and deployment system of claim 1, further comprising a tether element extending with respect to said implantable device to a location spaced from said implantable device, wherein said control handle comprises at least a tether adjustment system operably engageable with said tether element to adjust the tension on and/or length of said tether element between said implantable device and the location spaced from said implantable device.

7. The delivery and deployment system of claim 6, wherein said tether adjustment system comprises a fine tuning knob engageable with said tether element and rotatable about the longitudinal axis of said control handle to adjust the tension on and/or length of said tether element between said implantable device and the location spaced from said implantable device.

8. The delivery and deployment system of claim 7, further comprising a tensioning and locking device operably associated with said tether element and shiftable from a tension-adjusting configuration, in which tension on and/or the length of said tether element is adjustable, to a tension-setting configuration, in which tension on and/or the length of said tether element is fixed with respect to said implantable device.

9. The delivery and deployment system of claim 8, wherein said control handle further comprises a tensioning and locking system operably associated with said tensioning and locking device to shift said tensioning and locking device between the tension-adjusting configuration and the tension-setting configuration.

10. The delivery and deployment system of claim 9, wherein said tensioning and locking system includes a tensioning and locking knob rotatable about the longitudinal axis of said control handle to shift said tensioning and locking device between the tension-adjusting configuration and the tension-setting configuration.

11. The delivery and deployment system of claim 10, wherein said tensioning and locking system further comprises an anchor state indicator operably associated with said tensioning and locking knob to indicate the configuration of said tensioning and locking device.

12. The delivery and deployment system of claim 10, further comprising a stylet operably coupled with said implantable device.

13. The delivery and deployment system of claim 12, wherein said control handle comprises at least an implantable device deployment system operably coupled with said stylet to axially translate said stylet along the longitudinal axis of said control handle to advance and to deploy the implantable device from the implantable device delivery device.

14. The delivery and deployment system of claim 12, wherein said stylet is operably coupled with said tensioning and locking device and said tensioning and locking system such that rotation of said tensioning and locking knob rotates said stylet, and rotation of stylet shifts said tensioning and locking device between the tension-adjusting configuration and the tension-setting configuration.

15. The delivery and deployment system of claim 1, further comprising a tether element and a tensioning and locking device operably associated with said tether element, wherein:
said tether element extends from said implantable device to a location spaced from said implantable device;
said tensioning and locking device is shiftable from a tension-adjusting configuration, in which tension on and/or the length of said tether element is adjustable, to a tension-setting configuration, in which tension on and/or the length of said tether element is fixed with respect to said implantable device; and
said control handle comprises at least said tensioning and locking device to shift said tensioning and locking device between the tension-adjusting configuration and the tension-setting configuration.

16. A delivery and deployment system for delivering and/or deploying an implantable device to an anatomical site, said delivery and deployment system comprising a control handle having a longitudinal axis and comprising at least one control knob rotatable about the longitudinal axis of said control handle and operable to axially translate a component of said delivery and deployment system.

17. The delivery and deployment system of claim 16, wherein:
said delivery and deployment system includes a flexible elongate member configured to deliver an implantable device to an anatomical site transluminally; and
a pull element is operably coupled with said flexible elongate member and said control knob such that rotation of said control knob about the longitudinal axis of said control handle bends a distal region of said flexible elongate member to deliver the implantable device to a treatment site.

18. The delivery and deployment system of claim 16, wherein said control knob is rotatable about the longitudinal axis of said control handle to adjust an element of said delivery and deployment system with respect to a tether element operably associated with an implantable device deliverable by said delivery and deployment system.

19. A method of delivering and/or deploying an implantable device transluminally, said method comprising:
rotating a control knob operably associated with a delivery and deployment system control handle about the longitudinal axis of the control handle to axially translate an element of a delivery and deployment system associated with an implantable device.

20. The method of claim 19, wherein the axially translated element is at least one of a pull element operably associated with a steerable flexible elongated member and axially translatable to bend the steerable flexible elongate member to steer the steerable flexible elongate member within a patient's body to deliver an implantable device operably associated with the steerable flexible elongate member, or a tether element operably associated with an implantable device, or a flexible elongate member operably associated with a tether element and an implantable device to adjust tension on and/or the length of the tether element with respect to the implantable device.
